(19) United States
(12) Patent Application Publication
(10) Pub. No.: US 2021/0019921 A1
(43) Pub. Date: Jan. 21, 2021

(54) IMAGE PROCESSING DEVICE, IMAGE PROCESSING METHOD, AND PROGRAM

(71) Applicant: SONY CORPORATION, TOKYO (JP)

(72) Inventor: TAKUJI YOSHIDA, CHIBA (JP)

(21) Appl. No.: 17/040,034

(22) PCT Filed: Jan. 15, 2019
(86) PCT No.: PCT/JP2019/000859

§ 371 Date: Sep. 21, 2020

(30) Foreign Application Priority Data
Mar. 29, 2018 (JP) 2018-064068

(51) Int. Cl. G06T 11/00; G06F 3/0488; G06F 3/0484; H04N 5/232; G09G 3/20

(52) U.S. Cl. CPC G06T 11/001; G06F 3/04886; G06F 3/04847; A61B 1/04; G09G 3/20; G09G 2340/0464; G09G 2354/00; H04N 5/23229

(57) ABSTRACT

A display processing unit 15 detects an edge component by using an image signal generated by an imaging unit 12 and processed by a camera processing unit 13, and performs highlighting processing on a pixel whose edge component has a value greater than or equal to a predetermined value, or pixels in a predetermined range including the pixel. A control unit 20 changeably sets the size and the position of a processing target area on which the highlighting processing is performed, on the basis of an operation signal supplied from an operation unit 18, and the like. For example, the control unit 20 may set the size and the position of the processing target area on the basis of a size setting operation and a position setting operation using an operation key of the operation unit 18, or may set the size and the position of the processing target area depending on a touch panel operation on the operation unit 18. Moreover, the processing target area may be set by utilizing a subject recognition result using the image signal. Peaking processing can be performed in a desired image area, and focus confirmation can be easily performed.

### TECHNICAL FIELD

The present technology relates to an image processing device, an image processing method, and a program, and facilitates focus confirmation.

### BACKGROUND ART

Conventionally, for the purpose of focus confirmation during live view imaging, in Patent Document 1, peaking display is performed in which a focused contour of a subject and a peripheral portion thereof are made to have different colors and luminances in a live view image. Furthermore, in Patent Document 2, peaking display is performed when automatic focus adjustment processing is performed and the focus is in the locked state.

### CITATION LIST

### Patent Document

Patent Document 1: Japanese Patent Application Laid-Open No. 2007-060328

Patent Document 2: Japanese Patent Application Laid-Open No. 2013-157804

### SUMMARY OF THE INVENTION

### Problems to be Solved by the Invention

By the way, if peaking display is performed on a subject different from a subject to be imaged intended by a photographer, it is difficult to easily confirm a focus state of the subject to be imaged.

Thus, it is an object of the present technology to provide an image processing device, an image processing method, and a program that makes it possible to easily perform focus confirmation.

### Solutions to Problems

A first aspect of the present technology is

an image processing device including:

a display processing unit that performs highlighting processing on the basis of an edge component detected by using an image signal; and a processing area setting unit that sets at least a size or a position of a processing target area on which the highlighting processing is performed.

In the present technology, the display processing unit performs highlighting processing, that is, a peaking processing on, for example, a pixel whose edge component detected by using an image signal of a live view image has a value greater than or equal to a predetermined value, or pixels in a predetermined range including the pixel. Here, examples of the highlighting processing include processing of replacing a target edge component signal with a replacement signal (replacement color signal), and a signal change processing of changing the luminance and the saturation. Furthermore, the display processing unit may set the predetermined value on the basis of the processing target area. Furthermore, the display processing unit may detect the edge component by filter processing, and set a filter used for the filter processing on the basis of the processing target area.

The processing area setting unit sets the processing target area by adjusting the size and the position of the processing target area on which the highlighting processing is performed. For example, the size of the processing target area is set depending on a size setting operation using an operation key of an operation unit that generates an operation signal depending on a user operation, and the position of the processing target area is set depending on a position setting operation. Furthermore, in the operation unit, in a case where a touch panel is used that is provided on a display surface of a display unit that displays the image based on the image signal, the processing area setting unit performs setting of the size and the position of the processing target area on the basis of a start position and an end position of the user operation on the touch panel, and the size of the processing target area is set on the basis of the end position with the start position as a reference of the position of the processing target area, for example. Furthermore, the processing area setting unit may set the processing target area on the basis of a predetermined subject detected by subject recognition using the image signal. For example, the processing area setting unit sets, as the processing target area, an area including the whole of the predetermined subject detected. Furthermore, the processing area setting unit may set the position of the processing target area on the basis of a posture of the predetermined subject detected. Furthermore, the processing area setting unit may set the processing target area to be an area having a preset shape. Moreover, the processing area setting unit may set the predetermined subject depending on an imaging scene mode when the image signal is generated.

Furthermore, the display processing unit may change a signal level inside or outside the processing target area. Furthermore, the display processing unit may change a signal used for replacement in the highlighting processing depending on a current focal position with respect to a focus position, or may change the signal used for replacement in the highlighting processing depending on the edge component. Moreover, the display processing unit may set a color of the signal used for replacement in the highlighting processing depending on a color inside the processing target area. Furthermore, the display processing unit may perform the highlighting processing at a predetermined cycle. Moreover, an output unit may be further included that outputs, to an external device, the image signal subjected to the highlighting processing in the display processing unit.

A second aspect of the present technology is

an image processing method including:

performing highlighting processing, by a display processing unit, on the basis of an edge component detected by using an image signal, and

performing setting, by a processing area setting unit, of at least a size or a position of a processing target area on which the highlighting processing is performed.

A third aspect of the present technology is

a program that causes a computer to execute processing using an image signal,

the program causing the computer to execute:

a procedure of performing highlighting processing on the basis of an edge component detected by using an image signal; and

a procedure of setting at least a size or a position of a processing target area on which the highlighting processing is performed.

Note that, the program of the present technology is, for example, a program that can be provided to a general-purpose computer capable of executing various programs and codes, by a storage medium, a communication medium to be provided in a computer readable form, for example, a storage medium such as an optical disk, a magnetic disk, or a semiconductor memory, or a communication medium such as a network. By providing such a program in a computer readable form, processing is implemented according to the program on the computer.

### Effects of the Invention

According to the present technology, the highlighting processing is performed on the basis of the edge component detected by using the image signal. Furthermore, setting is performed of at least the size or the position of the processing target area on which the highlighting processing is performed. Thus, the peaking processing can be performed on a desired image area, and the focus confirmation can be easily performed. Note that, the advantageous effects described in this specification are merely examples, and the advantageous effects of the present technology are not limited to them and may include additional effects.

### BRIEF DESCRIPTION OF DRAWINGS

FIG. 1 is a diagram exemplifying a configuration of an imaging device.

FIG. 2 is a flowchart illustrating a peaking processing operation.

FIG. 3 is a diagram exemplifying a peaking area.

FIG. 4 is a flowchart illustrating a first example of peaking area setting processing.

FIG. 5 is a diagram illustrating a display unit and an operation unit provided in the imaging device.

FIG. 6 is a diagram for explaining operation of the first example of the peaking area setting processing.

FIG. 7 is a flowchart illustrating a second example of the peaking area setting processing.

FIG. 8 is a diagram for explaining operation of the second example of the peaking area setting processing.

FIG. 9 is a flowchart illustrating a third example of the peaking area setting processing.

FIG. 10 is a diagram for explaining operation of the third example of the peaking area setting processing.

FIG. 11 is a diagram exemplifying a case where a pupil is used as a subject recognition result.

FIG. 12 is a diagram schematically illustrating an overall configuration of an operation room system.

FIG. 13 is a diagram illustrating a display example of an operation screen on a centralized operation panel.

FIG. 14 is a diagram illustrating an example of a state of surgery to which the operation room system is applied.

FIG. 15 is a block diagram illustrating an example of a functional configuration of a camera head and a CCU illustrated in FIG. 14.

FIG. 16 is a diagram illustrating a peaking area setting example.

### MODE FOR CARRYING OUT THE INVENTION

The following is a description of embodiments for carrying out the present technology. Note that, the description will be made in the following order.

1. Configuration of Imaging Device

2. Peaking Processing

2-1. Peaking Processing Operation

2-2. Peaking Area Setting Processing

2-3. Display of Peaking Processing

3. Application Example

<1. Configuration of Imaging Device>

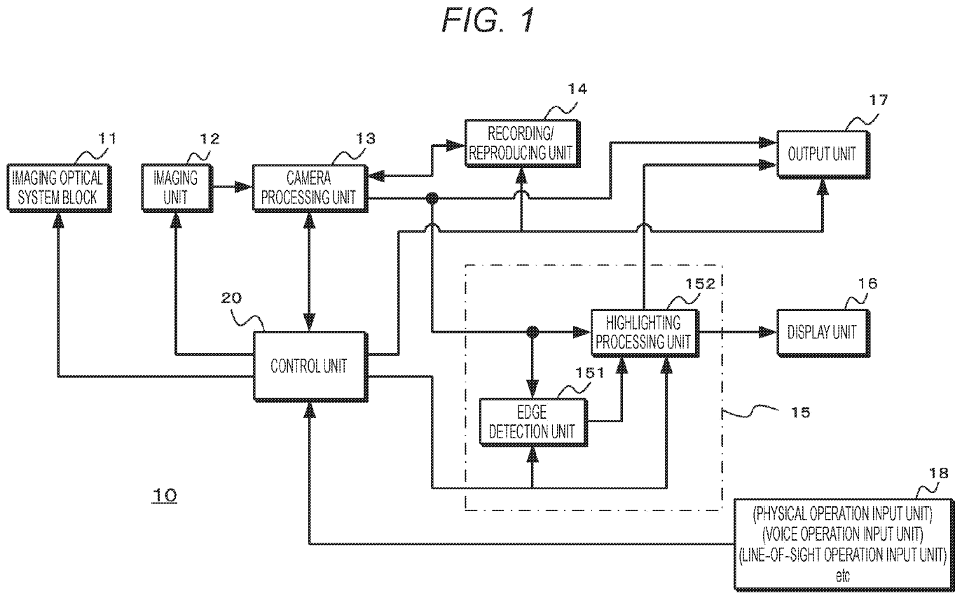

FIG. 1 exemplifies a configuration of an imaging device to which the technology according to the present disclosure is applied. An imaging device 10 includes an imaging optical system block 11, an imaging unit 12, a camera processing unit 13, a recording/reproducing unit 14, a display processing unit 15, a display unit 16, an output unit 17, an operation unit 18, and a control unit 20.

The imaging optical system block 11 includes a focus lens, a zoom lens, and the like. The imaging optical system block 11 drives the focus lens, the zoom lens, and the like on the basis of a control signal from the control unit 20 to form a subject optical image on the imaging surface of the imaging unit 12. Furthermore, the imaging optical system block 11 may be provided with an iris (aperture) mechanism, a shutter mechanism, and the like, and drive each mechanism on the basis of a control signal from the control unit 20.

The imaging unit 12 includes an imaging element such as a complementary metal oxide semiconductor (CMOS) or charge coupled device (CCD), and an element drive unit that drives the imaging element. The imaging unit 12 performs photoelectric conversion to generate an image signal depending on the subject optical image formed on the imaging surface of the imaging element. Furthermore, the imaging unit 12 performs noise removal processing, gain adjustment processing, analog/digital conversion processing, defective pixel correction, and the like on the image signal generated by the imaging element, and outputs the processed image signal to the camera processing unit 13.

The camera processing unit 13 performs processing such as gradation correction, color reproduction correction, edge enhancement, and gamma correction. Furthermore, in a case where a color mosaic filter is used in the imaging element of the imaging unit 12, the camera processing unit 13 performs demosaic processing to generate an image signal in which each pixel indicates each color of the color mosaic filter. The camera processing unit 13 converts the image signal after the camera processing into a luminance signal and a color difference signal and outputs the luminance signal and the color difference signal to the recording/reproducing unit 14. Furthermore, the camera processing unit 13 outputs the image signal after the camera processing to the display processing unit 15 and the output unit 17.

The recording/reproducing unit 14 converts the luminance signal and the color difference signal supplied from the camera processing unit 13 into those in a recording resolution and performs an encoding processing, and records obtained encoded signals in a predetermined file format on a recording medium (not illustrated). Furthermore, the recording/reproducing unit 14 performs decoding processing on the encoded signal read from the recording medium, and outputs the obtained luminance signal and color difference signal to the display processing unit 15. Furthermore, the recording/reproducing unit 14 may output the encoded signal to be recorded on the recording medium or the encoded signal recorded on the recording medium to the output unit 17.

The display processing unit 15 performs highlighting processing on a pixel whose value based on an edge component detected by using the image signal supplied from the camera processing unit 13 is greater than or equal to a predetermined value, or pixels in a predetermined range including the pixel.

The display processing unit 15 includes an edge detection unit 151 and a highlighting processing unit 152. The edge detection unit 151 uses the image signal supplied from the camera processing unit 13 to detect the edge component for each pixel in a processing target area (hereinafter referred to as “peaking area”) designated by the control unit 20. For example, the edge detection unit 151 performs high-pass (or band-pass) filter processing for each pixel by using the image signal in a target range, and generates an edge detection signal indicating the edge component. The edge detection unit 151 outputs the generated edge detection signal to the highlighting processing unit 152. Note that, the filter used to generate the edge detection signal may be a filter of a predetermined band, or may be a filter for which the band of the filter is set on the basis of the size of the peaking area. Specifically, the filter may be the one in which the band of the filter is set wide in a case where the size of the peaking area is greater than or equal to a predetermined size, or the one in which the band of the filter band is set narrow in a case where the size of the peaking area is less than or equal to the predetermined size. Moreover, the size of the peaking area and the band of the filter may be set to have a correlation. Furthermore, the edge detection unit 151 may generate an edge detection signal for a pixel other than the peaking area, and may output the edge detection signal corresponding to the peaking area among the generated edge detection signals to the highlighting processing unit 152.

The highlighting processing unit 152 performs highlighting processing on the basis of the edge detection signal generated by the edge detection unit 151. The highlighting processing unit 152 compares the edge detection signal supplied from the edge detection unit 151 with a preset threshold value, and changes a signal of a pixel whose signal level of the edge detection signal is greater than or equal to the threshold value to a preset replacement signal (replacement color signal). Furthermore, processing of the highlighting processing unit 152 is not limited to changing to the replacement signal as the highlighting processing, and may be processing of changing the luminance, saturation, or the like of the pixel whose signal level of the edge detection signal is greater than or equal to the threshold value. The highlighting processing unit 152 outputs the image signal after the highlighting processing to the output unit 17. Furthermore, the highlighting processing unit 152 converts the image signal after the highlighting processing into that of a display resolution and outputs the converted image signal to the display unit 16. Moreover, the highlighting processing unit 152 performs the highlighting processing in a predetermined operation mode on the basis of a control signal from the control unit 20, and in another operation mode, converts the image signal supplied from the camera processing unit 13 into that of the display resolution without performing the highlighting processing, and outputs the converted image signal to the display unit 16. Note that, similarly to the highlighting processing, the edge detection may be performed in the predetermined operation mode.

Furthermore, on the basis of the control signal from the control unit 20, the highlighting processing unit 152 may superimpose a display signal indicating a menu, various setting states, and the like on the image signal of the display resolution, and output the signal to the display unit 16.

The display unit 16 includes, for example, a liquid crystal display element or an organic EL display element. The display unit 16 displays a camera through image during imaging, a reproduced image recorded in a recording medium (not illustrated), and the like, by the image signal supplied from the display processing unit 15. Furthermore, the display unit 16 performs peaking display when being in the predetermined operation mode. The display unit 16 is provided on the back surface of the imaging device, for example. Alternatively, the display unit 16 may be provided as an electronic view finder, both a rear monitor and the electronic viewfinder may be individually provided, or the display unit 16 may be provided to be detachable to the imaging device 10. Note that, the display unit 16 may perform display of the menu, various setting states, and the like, and the operation unit 18 such as a touch panel may be provided on the screen of the display unit 16 as a graphical user interface (GUI).

The output unit 17 transmits at least one of the image signal supplied from the camera processing unit 13, the image signal supplied from the display processing unit 15, or the encoded signal supplied from the recording/reproducing unit 14 to an external device via a wireless or wired transmission path.

The operation unit 18 generates an operation signal depending on a user operation, and outputs the operation signal to the control unit 20. The operation unit 18 includes at least one of a plurality of operation input units, and the operation input unit generates an operation signal depending on the user operation. The plurality of operation input units is, for example, a physical operation input unit, a voice operation input unit, a line-of-sight operation input unit, or the like. In the physical operation input unit, operation is performed by using force applied by a user, with an operation key, an operation dial, an operation lever, the above-described touch panel, or the like. In the voice operation input unit, operation is performed by using a recognition result of a voice uttered by the user or the like. In the line-of-sight operation input unit, the line-of-sight of the user is recognized, and operation is performed by using at least one of the recognition results, for example, the position of the line-of-sight, the moving direction of the line-of-sight, the amount of movement of the line-of-sight, or the like.

The control unit 20 includes a central processing unit (CPU), read only memory (ROM), random access memory (RAM), and the like. The read only memory (ROM) stores various programs executed by the central processing unit (CPU). The random access memory (RAM) stores information such as various parameters. The CPU executes various programs stored in the ROM, and controls each unit so that the operation depending on the user operation is performed by the imaging device 10 on the basis of the operation signal from the operation unit 18. Furthermore, when being in the predetermined operation mode, for example, in an operation mode for displaying a live view image (live view operation mode), the control unit 20 controls peaking processing of the display processing unit 15 so that focus confirmation can be easily performed. For example, the control unit 20 sets the peaking area by adjusting the size and the position of the peaking area. For example, the control unit 20 sets the size and the position of the peaking area on which the peaking area is set on the basis of, for example, a subject recognition result obtained by performing subject recognition by using the operation signal or the image signal from the operation unit 18. Furthermore, the control unit 20 may set or change the threshold value used in the display processing unit 15, set or change the replacement color, or the like.

<2. Peaking Processing>

<2-1. Peaking Processing Operation>

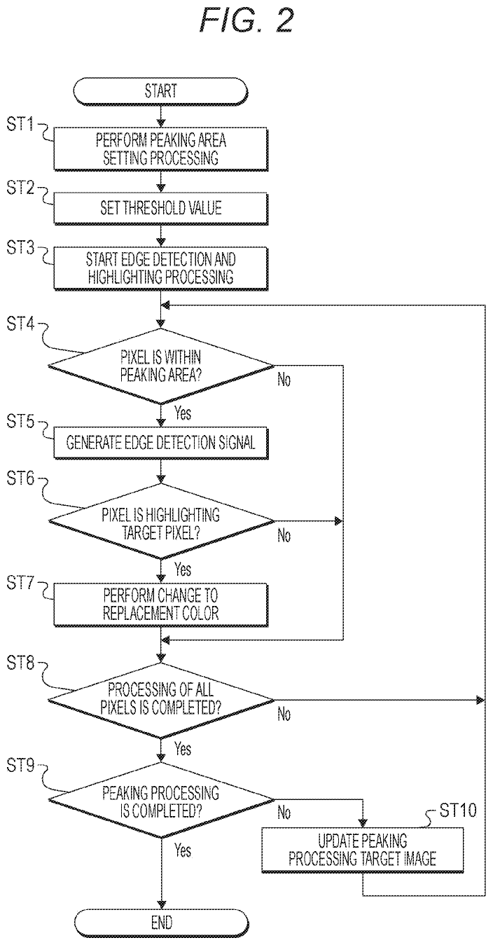

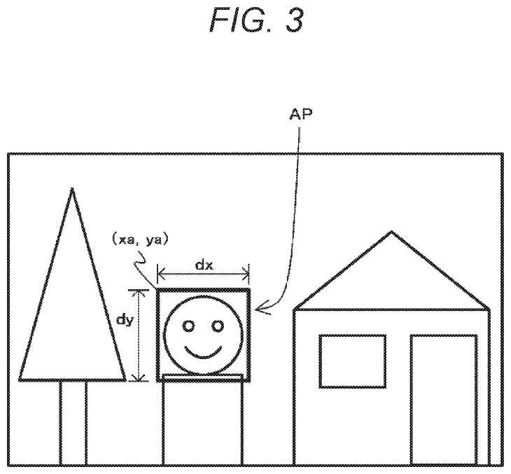

Next, a peaking processing operation performed by the imaging device 10 will be described. FIG. 2 is a flowchart illustrating the peaking processing operation. Furthermore, FIG. 3 exemplifies the peaking area.

In step ST1, the control unit performs peaking area setting processing. The control unit 20 sets the size (for example, a size dx in the horizontal direction and a size dy in the vertical direction) and the position (for example, a reference position (xa, ya) of the peaking area) of a peaking area AP as illustrated in FIG. 3 on the basis of a user operation or the like, and the processing proceed to step ST2. Details of the peaking area setting processing will be described later.

In step ST2, the control unit sets a threshold value. The control unit 20 sets a threshold value Eth for determining a pixel for which color replacement is performed on the basis of an edge detection signal. A preset value may be used as the threshold value, or a value used at the time of previous imaging may be used. Furthermore, the threshold value may be set, or the set threshold value may be changed depending on the user operation on the basis of the operation signal.

Furthermore, the threshold value may be set on the basis of a value of the generated edge detection signal. Specifically, an average value or a median value of the generated edge detection signals, or a value corresponding to a predetermined ratio from the higher order in a histogram generated is set as the threshold value. As a result, the peaking display is performed suitable for the value of the generated edge detection signal, and confirmation by the user is facilitated.

Furthermore, the threshold value may be set on the basis of the size of the set peaking area. Specifically, in a case where the size of the peaking area is less than or equal to the predetermined size, the threshold value is set high. In a case where the size of the peaking area is small, the user may intend to focus at a specific point such as a pinpoint, and when the threshold value is set so that the peaking display is performed in the entire peaking area, it is difficult to perform focusing, so that the threshold value is set high. Note that, the setting of the threshold value based on the size of the peaking area is not limited to a case where the threshold value is set high only in a case where the peaking area is small, and may be set so that the threshold value is set low in a case where the size of the peaking area is less than or equal to the predetermined size, the threshold value is set high or low in a case where the size of the peaking area is greater than or equal to the predetermined size, or the size of the peaking area and the threshold value are made to have a correlation.

The control unit 20 outputs a control signal indicating the set threshold value to the highlighting processing unit 152 of the display processing unit 15, and the processing proceeds to step ST3.

In step ST3, the control unit starts the edge detection and the highlighting processing. In a case where the imaging device 10 is in the predetermined operation mode, for example, the live view operation mode, the control unit 20 controls operation of the display processing unit 15 to start the edge detection and the highlighting processing, and the processing proceeds to step ST4.

In step ST4, the display processing unit determines whether or not the pixel is within the peaking area. The edge detection unit 151 of the display processing unit 15 uses the image signal output from the camera processing unit 13 in pixel order to determine whether or not the pixel is within the peaking area set in the processing of step ST1. In a case where the edge detection unit 151 determines that the pixel is within the peaking area, the processing proceeds to step ST5, and in a case where it is determined that the pixel is not within the peaking area, the processing proceeds to step ST8.

In step ST5, the display processing unit generates an edge detection signal. The edge detection unit 151 of the display processing unit 15 generates the edge detection signal for the pixel within the peaking area. For example, a processing target pixel for which the edge detection signal is generated is set as coordinates (i, j), and a signal level of the processing target pixel is set as P(i, j). In this case, the edge detection unit 151 performs calculation of the expression (1), generates an edge detection signal E(i, j) indicating the edge component, and the processing proceeds to step ST6.

E(i, j)=P(i, j)−(P(i−1, j)+2P(i, j)+P(i+1, j))/4 (1)

In step ST6, the display processing unit determines whether or not the pixel is a highlighting target pixel. The highlighting processing unit 152 of the display processing unit 15 compares the edge detection signal generated in step ST5 with the preset threshold value, and sets a pixel whose edge detection signal E(i, j) is greater than or equal to the threshold value Eth as the highlighting target pixel, and the processing proceeds to step ST7. Furthermore, to facilitate confirmation of the peaking display, the highlighting processing unit 152 of the display processing unit 15 may set pixels in a predetermined range including the determined highlighting target pixel as the highlighting target pixels, and in a case where an area of the highlighting target pixels is smaller than a predetermined size, the pixels in the predetermined range including the highlighting target pixel may be set as the highlighting target pixels. The highlighting processing unit 152 determines that a pixel whose edge detection signal E(i, j) is smaller than the threshold value Eth is not the highlighting target pixel, and the processing proceeds to step ST8.

In step ST7, the display processing unit performs change to the replacement color. The highlighting processing unit 152 of the display processing unit 15 changes the signal level P(i, j) of the pixel determined as the highlighting target pixel in step ST6 to a preset replacement color signal level Vpk, whereby the highlighting target pixel is identifiably displayed, and the processing proceeds to step ST8.

In step ST8, the display processing unit determines whether or not the processing is completed for all pixels in the image. The display processing unit 15 determines whether or not the processing of steps ST4 to ST7 is performed for each pixel in the image, and in a case where there is an unprocessed pixel, the processing returns to step ST4, and the processing of steps ST4 to ST7 is performed for unprocessed pixels. Furthermore, in a case where the display processing unit 15 determines that there is no unprocessed pixel, the processing proceeds to step ST9.

In step ST9, the control unit determines whether or not the peaking processing is ended. In a case where the predetermined operation mode is switched to another operation mode or in a case where the operation of the imaging device is ended, the control unit 20 ends the peaking processing. Furthermore, in a case where the operation mode is the predetermined operation mode, the processing proceeds to step ST10.

In step ST10, the display processing unit updates the peaking processing target image. The display processing unit 15 sets the next image supplied from the camera processing unit 13 as the peaking processing target, the processing returns to step ST4, and the processing of steps ST4 to ST8 is performed by using the image signal of the new image that is the peaking processing target.

As described above, according to the present technology, the size and the position of the peaking area can be freely set by the user. Thus, it is possible to prevent that the peaking display is performed on a subject different from a subject to be imaged intended by a photographer, or that in a case where the peaking display is performed when the focus is in the locked state, the focus accuracy is poor and the peaking display is performed on a subject different from a subject to be imaged. Thus, in a case where the user performs confirmation of focusing with the imaging device 10, the focus confirmation of the subject to be imaged by peaking, and framing of the angle of view and the subject not disturbed by the peaking can be simultaneously and easily performed.

<2-2. Peaking Area Setting Processing>

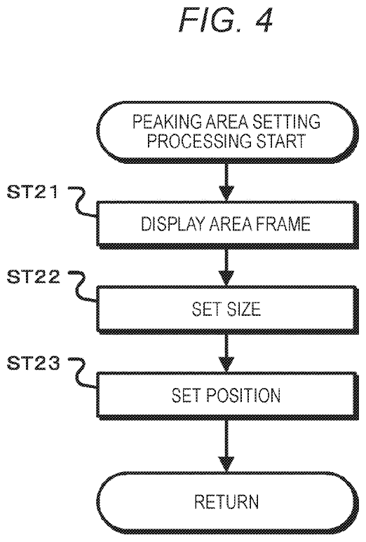

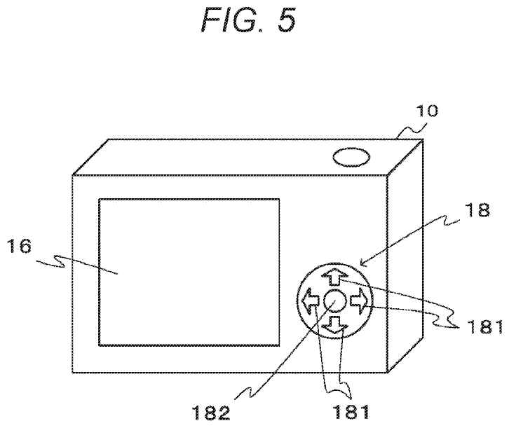

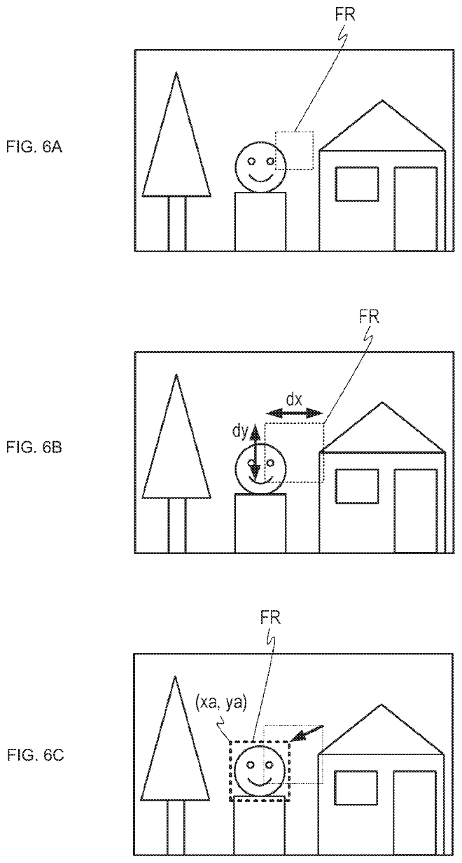

The control unit 20 sets a peaking area on the basis of a user operation or the like. In a first example of the peaking area setting processing, a case will be described in which the size and the position of the peaking area are individually set on the basis of the user operation. FIG. 4 is a flowchart illustrating the first example of the peaking area setting processing. Furthermore, FIG. 5 illustrates a display unit and an operation unit provided in the imaging device. The operation unit 18 includes, for example, a plurality of selection keys 181 and an enter key 182. FIG. 6 is a diagram for explaining operation of the first example of the peaking area setting processing.

In step ST21, the control unit displays an area frame indicating the peaking area. The control unit 20 displays an area frame FR indicating the peaking area as illustrated in (a) of FIG. 6 by using the display unit 16, for example, and the processing proceeds to step ST22.

In step ST22, the control unit sets the size of the peaking area. The control unit 20 sets a size (dx, dy) of the peaking area on the basis of an operation signal. The control unit 20 causes the display unit 16 to display a menu display for selecting a size from a plurality of area sizes. The user operates the selection key 181 illustrated in FIG. 5 to select a desired size, and operates the enter key 182. On the basis of the operation signal from the operation unit 18, the control unit 20 sets the size selected when the enter key is operated as the size of the peaking area, and as illustrated in (b) of FIG. 6, sets the area frame FR as the size (dx, dy) of the selected peaking area. Furthermore, the control unit 20 may change the size of the area frame FR depending on the user's pinch-in operation, pinch-out operation, or the like on the basis of the operation signal, and set the size of the area frame FR at the time of area size setting completion operation as the size (dx, dy) of the peaking area. Furthermore, the control unit 20 may automatically changes the size of the area frame FR in a case where the duration of touch operation exceeds a predetermined time on the basis of the operation signal, and set the size of the area frame FR at the end of the touch operation as the size (dx, dy) of the peaking area. Note that, the area size setting completion operation is not limited to the operation of setting the size of the displayed area frame FR as the size of the peaking area (for example, tap operation), but may include switching operation for setting the position of the peaking area, a predetermined time lapse after the size change, and the like. As described above, the control unit 20 sets the size of the peaking area depending on the user operation, and the processing proceeds to step ST23.

In step ST23, the control unit sets the position of the peaking area. The control unit 20 moves a display position of the area frame FR depending on the user operation for the selection key 181 illustrated in FIG. 5. Furthermore, when detecting that the enter key operation is performed, the control unit 20 sets the position of the displayed area frame FR as the position of the peaking area, and as illustrated in (c) of FIG. 6, sets the upper left position of the area frame FR as the reference position (xa, ya) of the peaking area. Furthermore, the control unit 20 may change the position of the area frame FR depending on the user's drag and drop operation or the like on the basis of the operation signal, and set, for example, the upper left position of the area frame FR at the time of area position setting completion operation as the reference position (xa, ya) of the peaking area. Note that, the area position setting completion operation is not limited to the operation of setting the position of the displayed area frame FR as the size of the peaking area (operation for a position determination button BS or tap operation), but may include a predetermined time lapse after the position change, and the like.

The control unit 20 performs processing illustrated in FIG. 4 to set the peaking area, and the processing proceeds to step ST2 in FIG. 2. Note that, in the processing illustrated in FIG. 4, the position of the peaking area may be set in step ST23 and then the size of the peaking area may be set in step ST22. Moreover, the control unit 20 may perform size adjustment and position adjustment of the area frame on the basis of the operation signal, and set the size and the position of the area frame FR at the time of operation of determining the size and the position of the peaking area as the size and the position of the peaking area.

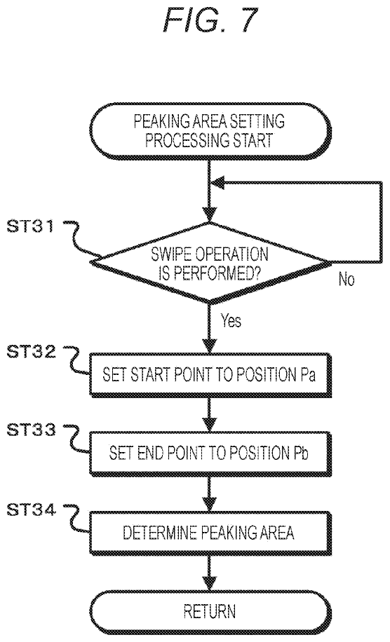

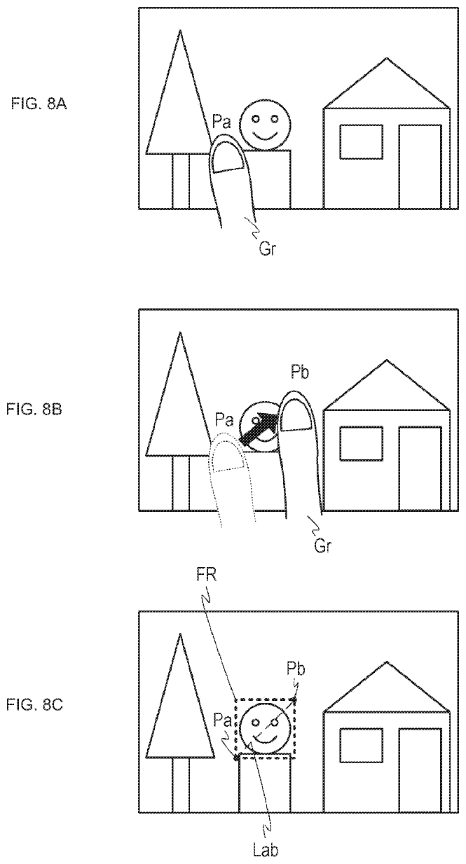

Next, in a second example of the peaking area setting processing, a case will be described in which the size and the position of the peaking area are set at the same time on the basis of a user operation. Note that, the operation unit 18 includes, for example, a touch panel provided on the display surface of the display unit 16 for displaying an image based on the image signal. FIG. 7 is a flowchart illustrating the second example of the peaking area setting processing. Furthermore, FIG. 8 is a diagram for explaining operation of the second example of the peaking area setting processing.

In step ST31, the control unit determines whether or not a swipe operation is performed. On the basis of the operation signal, the control unit 20 determines whether or not the swipe operation is performed in which the user touches the touch panel on the screen and slides a fingertip. In a case where the control unit 20 determines that the swipe operation is performed, the processing proceeds to step ST32, and in a case where it is not determined that the swipe operation is performed, the processing returns to step ST31. For example, when a finger Gr touching a position Pa illustrated in (a) of FIG. 8 is slid as illustrated by an arrow to a position Pb as illustrated in (b) of FIG. 8, the processing of step ST32 and subsequent steps is performed.

In step ST32, the control unit sets a start point of the swipe operation as the position Pa. The control unit 20 sets a fingertip position when the slide operation is started in step ST31 as the position Pa, and the processing proceeds to step ST33.

In step ST33, the control unit sets an end point of the swipe operation as the position Pb. The control unit 20 sets, as the position Pb, a fingertip position when the slide operation is ended that is determined as the swipe operation in step ST31, and the processing proceeds to step ST34.

In step ST34, the control unit determines the peaking area. The control unit 20 sets a rectangular area having a diagonal line connecting the position Pa set in step ST32 and the position Pb set in step ST33 together as the peaking area. As illustrated in (c) of FIG. 8, the control unit 20 sets, as a peaking area, a rectangular area having a straight line Lab connecting the position Pa at which the swipe operation is started and the position Pb at which the swipe operation is ended together as a diagonal line, and the peaking area is indicated by the area frame FR.

Furthermore, the peaking area is not limited to the rectangular shape. For example, in a case where the peaking area is set in a circular shape, a circular area whose diameter is a straight line connecting the position Pa and the position Pb together may be set as the peaking area, and the size of the peaking area may be set on the basis of the end position with the start position of the operation as a reference for the position of the peaking area. For example, a circular area having the position Pa as the center of the peaking area and the radius of a distance from the position Pa to the position Pb is set as the peaking area. Note that, the shape of the peaking area may be designated by the user or the like in advance before the peaking setting processing is performed, or may be designated by the user when the peaking setting processing is started. Furthermore, the control unit 20 may instruct the user to input the position Pa and the position Pb, for example, and set the peaking area as described above by using the position Pa and the position Pb designated by the touch operation. Furthermore, the setting of the peaking area is not limited to one, and a plurality of peaking areas may be set.

Next, in a third example of the peaking area setting processing, the peaking area is set on the basis of a predetermined subject detected by subject recognition using the image signal generated by the imaging unit 12. Furthermore, the predetermined subject detected by subject recognition may be set depending on the imaging scene mode when the image signal is generated by the imaging unit 12. The third example exemplifies a case where the imaging scene mode is set to, for example, the portrait mode, and a person's face is detected in subject recognition.

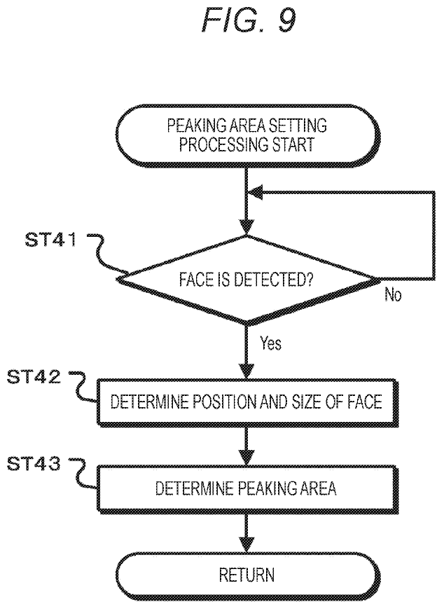

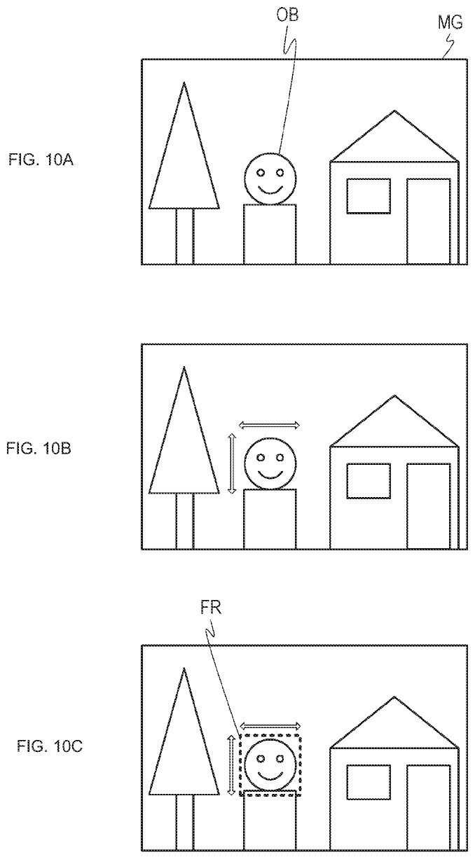

FIG. 9 is a flowchart illustrating the third example of the peaking area setting processing. Furthermore, FIG. 10 is a diagram for explaining operation of the third example of the peaking area setting processing.

In step ST41, the control unit determines whether or not a face is detected by subject recognition. The control unit 20 determines whether or not the face is detected, for example, on the basis of a subject recognition result supplied from the camera processing unit 13. In a case where the face is detected in the control unit 20, the processing proceeds to step ST42, and in a case where the face is not detected, the processing returns to step ST41. For example, in a case where a desired subject, for example, a face OB is detected in the subject recognition result using a captured image MG illustrated in (a) of FIG. 10, the processing of step ST42 and subsequent steps is performed.

In step ST42, the control unit determines the position and the size of the face. The control unit 20 determines the position and the size on the image of the detected face OB as illustrated in (b) of FIG. 10, and the processing proceeds to step ST43.

In step ST43, the control unit determines the peaking area. On the basis of the determination result of the position and the size of the detected face image, as illustrated in (c) of FIG. 10, the control unit 20 sets a region including the entire face, for example, a rectangular area in which the contour of the face fits as a peaking area, and the peaking area is indicated by the area frame FR.

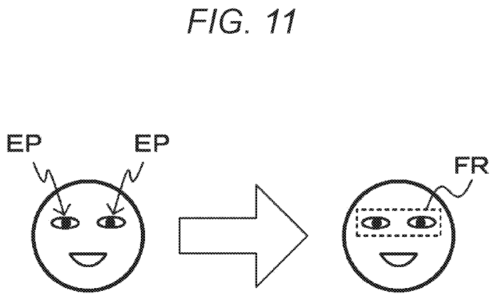

Furthermore, the control unit 20 may use, for example, a pupil as the subject recognition result. FIG. 11 is a diagram exemplifying a case where the pupil is used as the subject recognition result. In this case, the positions and the sizes of eyes are detected from the image on the basis of detected pupils EP, a rectangular area in which the detected eyes fit is set as a peaking area, and the peaking area is indicated by an area frame FR.

As described above, if the size and the position of the peaking area are automatically set by using the subject recognition result, without performing the setting operation for the size and the position of the peaking area, confirmation of focusing of the predetermined subject depending on the imaging scene mode, and framing of the angle of view and the subject not disturbed by the peaking can be simultaneously and easily performed.

Furthermore, the peaking area setting processing may be performed by using the voice operation input unit of the operation unit 18. In a voice operation, for example, words for setting the size of the peaking area (for example, “large”, “medium”, “small”, and the like), words for setting the position (for example, “up”, “down”, “right”, “left”, and the like), and words for designating the shape of the peaking area (for example, “square”, “circle”, and the like) are used. The voice operation input unit or the control unit 20 performs voice recognition using the voice signal collected by a microphone, determines which operation is performed, and the control unit 20 sets the peaking area depending on the determined operation.

As described above, if the peaking area setting processing is performed by using the voice, the user does not need to operate the operation key and the like, so that setting of the peaking area is facilitated, for example, in a case where confirmation of focusing or framing is performed by using the image displayed on a finder.

Furthermore, the peaking area setting processing may be performed by using the line-of-sight operation input unit of the operation unit 18. The line-of-sight operation input unit recognizes the line-of-sight of the user, determines which position on the image is targeted by the user on the basis of the recognition result, and uses the determined target position for setting the peaking area. For example, the peaking area can be set by using the determined two target positions as the positions Pa and Pb described in the second example.

Moreover, the control unit 20 can set the peaking area by combining the subject recognition result and the user operation. For example, the peaking area is set by using the subject recognition result and the operation signal from the physical operation input unit. In this case, the peaking area can be set to correspond to a face designated by the user operation from, for example, a plurality of faces detected by the subject recognition. Furthermore, the peaking area is set by using the subject recognition result and the operation signal from the voice operation input unit. In this case, for example, the peaking area can be set to correspond to a face part designated by the voice for the face detected by the subject recognition.

<2-3. Display of Peaking Processing>

Next, display of the peaking processing will be described. In the peaking area setting processing, the peaking area is indicated by using the area frame in the above-described embodiment, but the size and the position of the peaking area may be made identifiable by changing the signal level in the area inside or outside the peaking area. For example, the control unit 20 controls the display processing unit 15 to replace the image in the peaking area, to make a monochrome image, or an emphasized image with enhanced contrast. Furthermore, the control unit 20 controls the display processing unit 15 to replace the image outside the peaking area, to make a monochrome image or a fade image with reduced contrast. Thus, in the peaking area setting processing, the size and the position of the peaking area can be easily grasped also by replacing the image inside the peaking area or the image outside the peaking area.

Regarding the peaking display, in the above-described embodiment, the highlighting target pixel is replaced with a predetermined replacement color signal; however, the signal (replacement signal) used for replacement in the highlighting processing may be changed depending on the current focal position with respect to the focus position or the edge component detected by edge detection.

For example, in a case where the imaging unit is provided with a focus detection function indicating a focusing direction (for example, in a case where the imaging element is provided with an image plane phase difference sensor), the control unit 20 controls the highlighting processing unit 152 of the display processing unit 15 on the basis of a focus detection signal acquired from the imaging unit 12, to change the color depending on the current focal position with respect to the focus position. Specifically, the color is switched depending on whether the focus position is in a front focus in which the focus position is in front of the subject, or is a rear focus in which the focus position is in the back of the subject, and a warm color is set when in the front focus, and a cold color is set when in the rear focus. By changing the color of the replacement color in this way, a positional relationship between the focus position and the subject can be grasped by the peaking display.

Furthermore, in a case where the signal used for replacement in the highlighting processing is changed depending on the edge component, for example, the highlighting processing unit 152 of the display processing unit 15 changes the brightness of the replacement color depending on the edge component to cause the brightness to be increased as the edge component increases. Furthermore, the color of the replacement color may be changed depending on the edge component to cause the color to be changed from the cold color to the warm color as the edge component increases. By changing the replacement color in this way, the edge component can be roughly grasped.

Moreover, the control unit 20 may change the color of the signal used for replacement in the highlighting processing on the basis of a color in the peaking area. For example, with respect to an average color in the peaking area or an average color of a desired subject in the peaking area, an inverse hue (complementary color) or a color having a predetermined hue difference is set as the replacement color. By changing the replacement color in this way, a difference between the color in the peaking area and the color in the peaking display is remarkable, and the peaking display can be easily recognized.

Furthermore, the control unit 20 may control the operation of the display processing unit 15 to perform the highlighting processing at a predetermined cycle. In this case, since the peaking display is displayed intermittently, an original image in the peaking display area can be confirmed. Furthermore, the control unit 20 may control the operation of the display processing unit 15 to display a captured image on which the peaking display is performed and a captured image on which the peaking display is not performed side by side so that the original image in the peaking display area can be confirmed.

Moreover, the control unit 20 may control the operations of the display processing unit 15 and the output unit 17 to display the captured image on which the peaking display is performed on one or both of the display unit 16 and the external device. For example, in a case where focus control is performed by the external device, the captured image on which the peaking display is performed is output from the display processing unit 15 to the external device via the output unit 17. At this time, if the captured image on which the peaking display is not performed is displayed on the display unit 16, it is possible to perform various types of work on the basis of the captured image displayed on the display unit 16 without being affected by the peaking display.

<3. Application Example>

The technology according to the present disclosure can be applied to various products. For example, the technology according to the present disclosure may be applied to an operation room system.

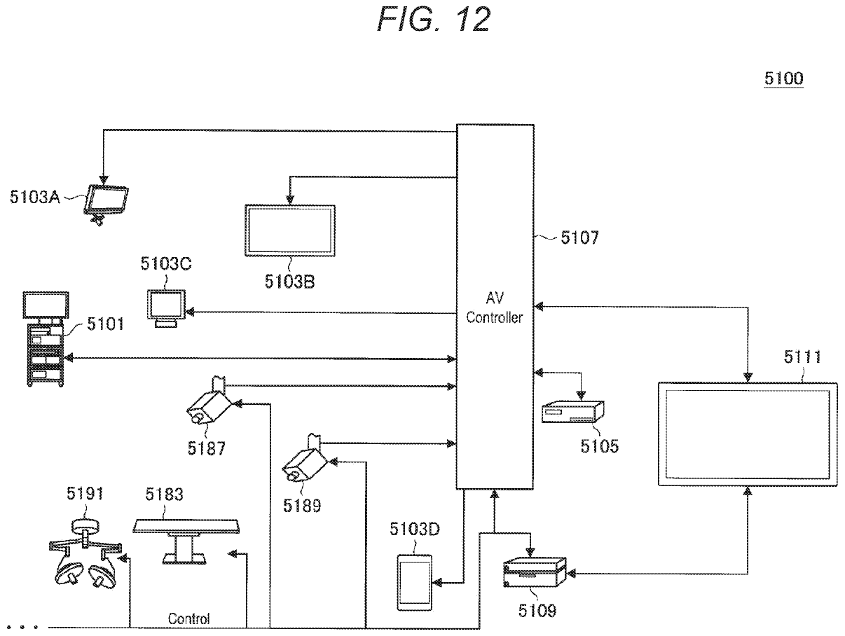

FIG. 12 is a diagram schematically illustrating an overall configuration of an operation room system 5100 to which the technology according to the present disclosure can be applied. Referring to FIG. 12, in the operation room system 5100, devices installed in an operation room are connected to each other to be able to cooperate with each other via an audiovisual controller (AV controller) 5107 and an operation room control device 5109.

Various devices can be installed in the operation room. FIG. 12 illustrates, as an example, various devices 5101 for endoscopic surgery, a ceiling camera 5187 provided on the ceiling of the operation room and imaging an area at hand of a surgeon, an operation room camera 5189 provided on the ceiling of the operation room and imaging a state of the entire operation room, a plurality of display devices 5103A to 5103D, a recorder 5105, a patient bed 5183, and an illumination 5191.

Here, among these devices, the devices 5101 belong to an endoscopic surgical system 5113 described later, and includes an endoscope, a display device that displays an image captured by the endoscope, and the like. Each device belonging to the endoscopic surgical system 5113 is also referred to as a medical device. On the other hand, the display devices 5103A to 5103D, the recorder 5105, the patient bed 5183, and the illumination 5191 are devices provided in, for example, the operation room, separately from the endoscopic surgical system 5113. Each device that does not belong to the endoscopic surgical system 5113 is also referred to as a non-medical device. The audiovisual controller 5107 and/or the operation room control device 5109 control operations of these medical devices and non-medical devices in cooperation with each other.

The audiovisual controller 5107 comprehensively controls processing regarding image display in the medical devices and non-medical devices. Specifically, among the devices included in the operation room system 5100, the devices 5101, the ceiling camera 5187, and the operation room camera 5189 each can be a device (hereinafter also referred to as a transmission source device) having a function of transmitting information (hereinafter also referred to as display information) to be displayed during surgery. Furthermore, the display devices 5103A to 5103D each can be a device (hereinafter also referred to as an output destination device) to which the display information is output. Furthermore, the recorder 5105 can be a device corresponding to both the transmission source device and the output destination device. The audiovisual controller 5107 has functions of controlling operations of the transmission source device and the output destination device, to acquire the display information from the transmission source device and transmit the display information to the output destination device for display or recording. Note that, the display information is various images captured during the surgery, and various types of information regarding the surgery (for example, patient's physical information, the past examination results, information about a surgical method, and the like) and the like.

Specifically, information about the image of the surgical portion in a body cavity of the patient captured by the endoscope is transmitted as display information from the devices 5101 to the audiovisual controller 5107. Furthermore, information about the image of the area at hand of the surgeon captured by the ceiling camera 5187 can be transmitted as display information from the ceiling camera 5187. Furthermore, information about the image indicating the state of the entire operation room captured by the operation room camera 5189 can be transmitted as display information from the operation room camera 5189. Note that, in a case where there is another device having an imaging function in the operation room system 5100, the audiovisual controller 5107 may acquire information about an image captured by the other device from the other device, as display information.

Alternatively, for example, information about these images captured in the past is recorded in the recorder 5105 by the audiovisual controller 5107. The audiovisual controller 5107 can acquire information about the image captured in the past from the recorder 5105 as display information. Note that, various types of information regarding surgery may also be recorded in advance in the recorder 5105.

The audiovisual controller 5107 causes at least one of the display devices 5103A to 5103D that are output destination devices to display the acquired display information (in other words, images captured during the surgery, and various types of information regarding the surgery). In the illustrated example, the display device 5103A is a display device installed to be suspended from the ceiling of the operation room, the display device 5103B is a display device installed on the wall of the operation room, the display device 5103C is a display device installed on a desk in the operation room, and the display device 5103D is a mobile device (for example, a tablet personal computer (PC)) having a display function.

Furthermore, although illustration is omitted in FIG. 12, the operation room system 5100 may include devices outside the operation room. The devices outside the operation room can be, for example, a server connected to a network built inside and outside a hospital, a PC used by a medical staff, a projector installed in a conference room of the hospital, and the like. In a case where such an external device is outside the hospital, the audiovisual controller 5107 can also cause a display device of another hospital to display the display information via a video conference system or the like, for telemedicine.

The operation room control device 5109 comprehensively controls processing other than the processing regarding the image display in the non-medical devices. For example, the operation room control device 5109 controls drive of the patient bed 5183, the ceiling camera 5187, the operation room camera 5189, and the illumination 5191.

A centralized operation panel 5111 is provided in the operation room system 5100, and a user can give an instruction about image display to the audiovisual controller 5107 via the centralized operation panel 5111, or an instruction about operation of the non-medical device to the operation room control device 5109. The centralized operation panel 5111 is configured as a touch panel provided on the display surface of the display device.

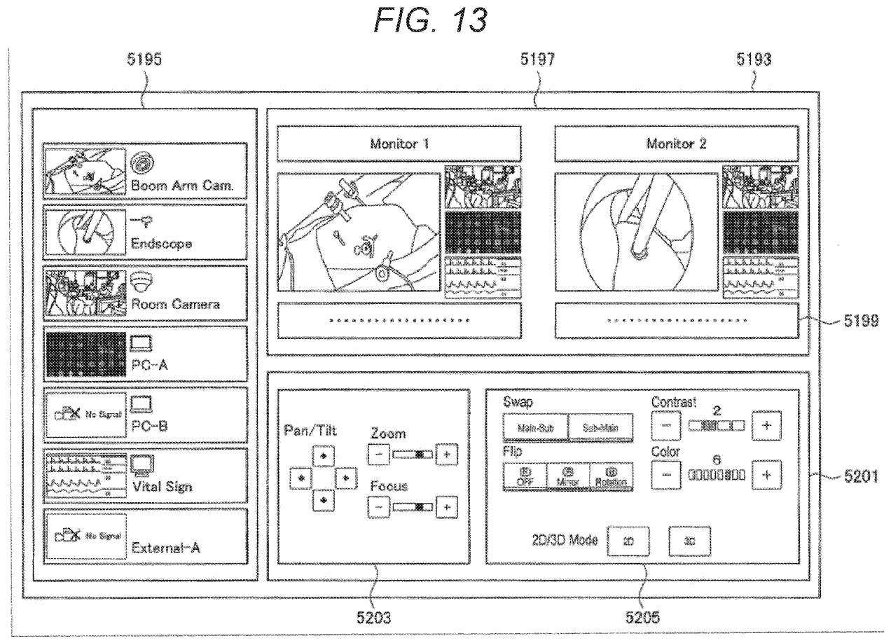

FIG. 13 is a diagram illustrating a display example of an operation screen on the centralized operation panel 5111. In FIG. 13, as an example, the operation screen is illustrated corresponding to a case where the operation room system 5100 is provided with two display devices as the output destination devices. Referring to FIG. 13, an operation screen 5193 is provided with a transmission source selection area 5195, a preview area 5197, and a control area 5201.

In the transmission source selection area 5195, the transmission source devices included in the operation room system 5100 and respective thumbnail screens representing the display information of the transmission source devices are displayed in association with each other. The user can select the display information to be displayed on the display device from any of the transmission source devices displayed in the transmission source selection area 5195.

In the preview area 5197, previews are displayed of screens displayed on the respective two display devices (Monitor 1 and Monitor 2) that are output destination devices. In the illustrated example, four images are PinP-displayed in one display device. The four images correspond to the display information transmitted from the transmission source device selected in the transmission source selection area 5195. Among the four images, one is displayed relatively large as a main image, and the remaining three are displayed relatively small as sub-images. The user can switch the main image and the sub-images with each other by appropriately selecting one of four areas in which the respective images are displayed. Furthermore, a status display area 5199 is provided below an area in which the four images are displayed, and a status regarding the surgery (for example, an elapsed time of the surgery, the patient's physical information, and the like) is displayed in the area as appropriate.

The control area 5201 is provided with a transmission source operation area 5203 in which graphical user interface (GUI) components are displayed for performing operation to the transmission source device, and an output destination operation area 5205 in which GUI components are displayed for performing operation to the output destination device. In the illustrated example, in the transmission source operation area 5203, the GUI components are provided for performing various operations (pan, tilt, and zoom) to a camera in the transmission source device having an imaging function. The user can operate the operation of the camera in the transmission source device by appropriately selecting these GUI components. Note that, although not illustrated, in a case where the transmission source device selected in the transmission source selection area 5195 is a recorder (in other words, in a case where an image recorded in the recorder in the past is displayed on the preview area 5197), a GUI component for performing operations such as reproduction, reproduction stop, rewind, and fast-forward of the image can be provided in the transmission source operation area 5203.

Furthermore, in the output destination operation area 5205, the GUI components are provided for performing various operations (swap, flip, color adjustment, contrast adjustment, switching between 2D display and 3D display) to a display on the display device that is the output destination device. The user can operate the display on the display device by appropriately selecting these GUI components.

Note that, the operation screen displayed on the centralized operation panel 5111 is not limited to the illustrated example, and the user may be capable of operation input to each device that may be controlled by the audiovisual controller 5107 and the operation room control device 5109 included in the operation room system 5100 via the centralized operation panel 5111.

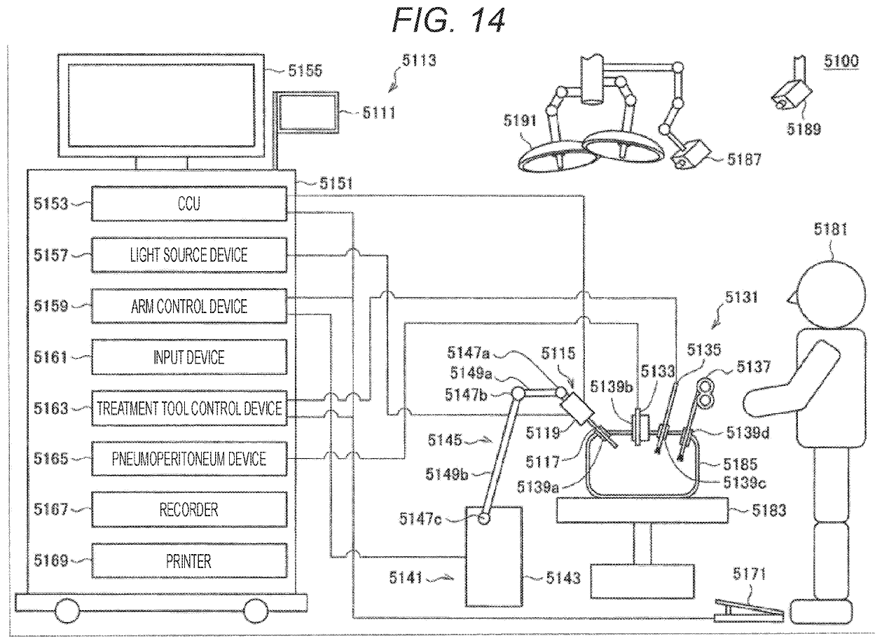

FIG. 14 is a diagram illustrating an example of a state of surgery to which the operation room system described above is applied. The ceiling camera 5187 and the operation room camera 5189 are provided on the ceiling of the operation room, and can image the state of the area at hand of a surgeon (surgeon) 5181 who performs treatment on an affected part of a patient 5185 on the patient bed 5183, and the entire operation room. The ceiling camera 5187 and the operation room camera 5189 can be provided with a magnification adjustment function, a focal length adjustment function, an imaging direction adjustment function, and the like. The illumination 5191 is provided on the ceiling of the operation room, and irradiates at least the area at hand of the surgeon 5181. The illumination 5191 may be enabled to appropriately adjust the amount of irradiation light, the wavelength (color) of the irradiation light, the irradiation direction of the light, and the like.

As illustrated in FIG. 12, the endoscopic surgical system 5113, the patient bed 5183, the ceiling camera 5187, the operation room camera 5189, and the illumination 5191 are connected to each other to be able to cooperate with each other via the audiovisual controller 5107 and the operation room control device 5109 (not illustrated in FIG. 14). The centralized operation panel 5111 is provided in the operation room, and as described above, the user can appropriately operate these devices existing in the operation room via the centralized operation panel 5111.

Hereinafter, a configuration of the endoscopic surgical system 5113 will be described in detail. As illustrated, the endoscopic surgical system 5113 includes an endoscope 5115, other surgical tools 5131, a support arm device 5141 that supports the endoscope 5115, and a cart 5151 on which various devices for endoscopic surgery are mounted.

In endoscopic surgery, instead of performing laparotomy by incising an abdominal wall, a plurality of cylindrical opening devices called trocars 5139a to 5139d punctures the abdominal wall. Then, a lens barrel 5117 of the endoscope 5115 and the other surgical tools 5131 are inserted into a body cavity of the patient 5185 from the trocars 5139a to 5139d. In the illustrated example, a pneumoperitoneum tube 5133, an energy treatment tool 5135, and forceps 5137 are inserted into the body cavity of the patient 5185 as the other surgical tools 5131. Furthermore, the energy treatment tool 5135 is a treatment tool that performs incision and peeling of tissue, sealing of a blood vessel, or the like by a high-frequency current or ultrasonic vibration. However, the surgical tools 5131 illustrated are merely examples, and various surgical tools generally used in endoscopic surgery may be used as the surgical tools 5131, for example, tweezers, a retractor, and the like.

An image of a surgical portion in the body cavity of the patient 5185 imaged by the endoscope 5115 is displayed on a display device 5155. The surgeon 5181 performs a treatment, for example, excising the affected part, or the like, by using the energy treatment tool 5135 and the forceps 5137 while viewing the image of the surgical portion displayed on the display device 5155 in real time. Note that, although not illustrated, the pneumoperitoneum tube 5133, the energy treatment tool 5135, and the forceps 5137 are supported by the surgeon 5181, an assistant, or the like during the surgery.

(Support Arm Device)

The support arm device 5141 includes an arm 5145 extending from a base 5143. In the illustrated example, the arm 5145 includes joints 5147a, 5147b, and 5147c and links 5149a and 5149b, and is driven by control of an arm control device 5159. The endoscope 5115 is supported by the arm 5145, and its position and posture are controlled. As a result, stable position fixing can be implemented of the endoscope 5115.

(Endoscope)

The endoscope 5115 includes the lens barrel 5117 in which a region of a predetermined length from the distal end is inserted into the body cavity of the patient 5185, and a camera head 5119 connected to the proximal end of the lens barrel 5117. In the illustrated example, the endoscope 5115 formed as a so-called rigid scope including the rigid lens barrel 5117 is illustrated, but the endoscope 5115 may be formed as a so-called flexible scope including the flexible lens barrel 5117.

At the distal end of the lens barrel 5117, an opening is provided into which an objective lens is fitted. A light source device 5157 is connected to the endoscope 5115, and light generated by the light source device 5157 is guided to the distal end of the lens barrel by a light guide extending inside the lens barrel 5117, and the light is emitted toward an observation target in the body cavity of the patient 5185 via the objective lens. Note that, the endoscope 5115 may be a forward-viewing endoscope, an oblique-viewing endoscope, or a side-viewing endoscope.

An optical system and an imaging element are provided inside the camera head 5119, and reflected light (observation light) from the observation target is focused on the imaging element by the optical system. The observation light is photoelectrically converted by the imaging element, and an electric signal corresponding to the observation light, that is, an image signal corresponding to the observation image is generated. The image signal is transmitted as RAW data to a camera control unit (CCU) 5153. Note that, in the camera head 5119, a function is installed of adjusting the magnification and the focal length by appropriately driving the optical system.

Note that, for example, to cope with stereoscopic vision (3D display) or the like, the camera head 5119 may be provided with a plurality of the imaging elements. In this case, a plurality of relay optical systems is provided inside the lens barrel 5117 to guide the observation light to each of the plurality of imaging elements.

(Various Devices Mounted on Cart)

The CCU 5153 includes a central processing unit (CPU), a graphics processing unit (GPU), and the like, and comprehensively controls operation of the endoscope 5115 and the display device 5155. Specifically, the CCU 5153 performs, on the image signal received from the camera head 5119, various types of image processing for displaying an image based on the image signal, for example, development processing (demosaic processing), and the like. The CCU 5153 provides the display device 5155 with the image signal on which the image processing is performed. Furthermore, the audiovisual controller 5107 illustrated in FIG. 12 is connected to the CCU 5153. The CCU 5153 also provides the audiovisual controller 5107 with the image signal on which the image processing is performed. Furthermore, the CCU 5153 transmits a control signal to the camera head 5119 to control its drive. The control signal can include information regarding imaging conditions such as the magnification and the focal length. The information regarding the imaging conditions may be input via an input device 5161, or may be input via the centralized operation panel 5111 described above.

The display device 5155 displays an image based on the image signal subjected to the image processing by the CCU 5153, by the control from the CCU 5153. In a case where the endoscope 5115 is compatible with high-resolution imaging, for example, 4K (the number of horizontal pixels 3840×the number of vertical pixels 2160), 8K (the number of horizontal pixels 7680×the number of vertical pixels 4320), and the like, and/or in a case where the endoscope 5115 is compatible with 3D display, as the display device 5155, corresponding to each case, a display device can be used capable of high-resolution display and/or 3D display. In a case where the display device 5155 is compatible with the high-resolution imaging such as 4K or 8K, a more immersive feeling can be obtained by using a display device having a size of greater than or equal to 55 inches. Furthermore, a plurality of the display devices 5155 having different resolutions and sizes may be provided depending on applications.

The light source device 5157 includes a light source, for example, a light emitting diode (LED) or the like, and supplies irradiation light for imaging a surgical portion to the endoscope 5115.

The arm control device 5159 includes a processor, for example, a CPU or the like, and controls drive of the arm 5145 of the support arm device 5141 in accordance with a predetermined control method by operating in accordance with a predetermined program.

The input device 5161 is an input interface to the endoscopic surgical system 5113. The user can input various types of information and instructions to the endoscopic surgical system 5113 via the input device 5161. For example, the user inputs various types of information regarding the surgery, such as the patient's physical information and information about the surgical method, via the input device 5161. Furthermore, for example, the user inputs, via the input device 5161, an instruction to drive the arm 5145, an instruction to change the imaging conditions (type of irradiation light, magnification, focal length, and the like) by the endoscope 5115, an instruction to drive the energy treatment tool 5135, and the like.

The type of the input device 5161 is not limited, and the input device 5161 may be any of various known input devices. As the input device 5161, for example, a mouse, a keyboard, a touch panel, a switch, a foot switch 5171 and/or a lever and the like can be applied. In a case where a touch panel is used as the input device 5161, the touch panel may be provided on the display surface of the display device 5155.

Alternatively, the input device 5161 is a device worn by the user, for example, a glasses-type wearable device, a head mounted display (HMD), or the like, and various inputs are performed depending on the user's gesture and line-of-sight detected by these devices. Furthermore, the input device 5161 includes a camera enabled to detect the user's movement, and various inputs are performed depending on the user's gesture and line-of-sight detected from a video captured by the camera. Moreover, the input device 5161 includes a microphone enabled to pick up a user's voice, and various inputs are performed by voice via the microphone. As described above, the input device 5161 is enabled to input various information without contact, whereby in particular the user (for example, the surgeon 5181) belonging to a clean area can operate a device belonging to an unclean area without contact. Furthermore, since the user can operate the device without releasing the user's hand from the surgical tool, convenience of the user is improved.

A treatment tool control device 5163 controls drive of the energy treatment tool 5135 for cauterization of tissue, incision, sealing of blood vessels, or the like. A pneumoperitoneum device 5165 injects a gas into the body cavity of the patient 5185 via the pneumoperitoneum tube 5133 to inflate the body cavity, for the purpose of securing a visual field by the endoscope 5115 and securing a working space of the surgeon. A recorder 5167 is a device enabled to record various types of information regarding surgery. A printer 5169 is a device enabled to print various types of information regarding surgery in various formats such as text, image, graph, and the like.

Hereinafter, a particularly characteristic configuration in the endoscopic surgical system 5113 will be described in detail.

(Support Arm Device)

The support arm device 5141 includes the base 5143 that is a base, and the arm 5145 extending from the base 5143. In the illustrated example, the arm 5145 includes the plurality of joints 5147a, 5147b, and 5147c, and the plurality of links 5149a and 5149b coupled together by the joint 5147b, but in FIG. 14, for simplicity, the configuration of the arm 5145 is simplified and illustrated. Actually, the shape, number, and arrangement of the joints 5147a to 5147c and the links 5149a and 5149b, the direction of the rotation axis of the joints 5147a to 5147c, and the like can be appropriately set so that the arm 5145 has a desired degree of freedom. For example, the arm 5145 can suitably have six degrees of freedom or more. As a result, the endoscope 5115 can be freely moved within the movable range of the arm 5145, so that the lens barrel 5117 of the endoscope 5115 can be inserted into the body cavity of the patient 5185 from a desired direction.

The joints 5147a to 5147c each are provided with an actuator, and the joints 5147a to 5147c each are rotatable around a predetermined rotation axis by drive of the actuator. The drive of the actuator is controlled by the arm control device 5159, whereby the rotation angle of each of the joints 5147a to 5147c is controlled, and the drive of the arm 5145 is controlled. As a result, control of the position and posture of the endoscope 5115 can be implemented. At this time, the arm control device 5159 can control the drive of the arm 5145 by various known control methods such as force control or position control.

For example, the surgeon 5181 performs operation input appropriately via the input device 5161 (including the foot switch 5171), whereby the drive of the arm 5145 may be appropriately controlled by the arm control device 5159 depending on the operation input, and the position and posture of the endoscope 5115 may be controlled. By the control, the endoscope 5115 at the distal end of the arm 5145 can be moved from an arbitrary position to an arbitrary position, and then fixedly supported at the position after the movement. Note that, the arm 5145 may be operated by a so-called master slave method. In this case, the arm 5145 can be remotely operated by the user via the input device 5161 installed at a location away from the operation room.

Furthermore, in a case where force control is applied, the arm control device 5159 may perform so-called power assist control in which external force is received from the user, and the actuator of each of the joints 5147a to 5147c is driven so that the arm 5145 moves smoothly following the external force. As a result, when the user moves the arm 5145 while directly touching the arm 5145, the arm 5145 can be moved with a relatively light force. Thus, the endoscope 5115 can be moved more intuitively and with a simpler operation, and the convenience for the user can be improved.

Here, in general, in the endoscopic surgery, the endoscope 5115 is supported by a surgeon called a scopist. In contrast, by using the support arm device 5141, the position of the endoscope 5115 can be more reliably fixed without relying on human hands, so that an image of the surgical portion can be stably obtained, and the surgery can be smoothly performed.

Note that, the arm control device 5159 is not necessarily provided in the cart 5151. Furthermore, the arm control device 5159 does not necessarily have to be one device. For example, the arm control device 5159 may be provided at each of the joints 5147a to 5147c of the arm 5145 of the support arm device 5141, and a plurality of the arm control devices 5159 cooperates with each other, whereby drive control of the arm 5145 may be implemented.

(Light Source Device)

The light source device 5157 supplies the endoscope 5115 with irradiation light when a surgical portion is imaged. The light source device 5157 includes a white light source including, for example, an LED, a laser light source, or a combination thereof. At this time, in a case where the white light source includes a combination of R, G, and B laser light sources, the output intensity and the output timing of each color (each wavelength) can be controlled with high accuracy, so that adjustment can be performed of the white balance of the captured image in the light source device 5157. Furthermore, in this case, it is also possible to capture an image corresponding to each of R, G, and B in time division by emitting the laser light from each of the R, G, and B laser light sources in time division to the observation target, and controlling drive of the imaging element of the camera head 5119 in synchronization with the emission timing. According to this method, a color image can be obtained without providing a color filter in the imaging element.

Furthermore, drive of the light source device 5157 may be controlled such that the intensity of light to be output is changed at predetermined time intervals. By controlling the drive of the imaging element of the camera head 5119 in synchronization with the change timing of the light intensity to acquire images in time division, and synthesizing the images, a high dynamic range image can be generated without so-called blocked up shadows or blown out highlights.

Furthermore, the light source device 5157 may be able to supply light of a predetermined wavelength band corresponding to special light observation. In the special light observation, for example, by using wavelength dependence of light absorption in a body tissue, by emitting narrow band light compared to irradiation light (in other words, white light) at the time of ordinary observation, so-called narrow band imaging is performed in which a predetermined tissue such as a blood vessel in a mucosal surface layer is imaged with high contrast. Alternatively, in the special light observation, fluorescence observation may be performed that obtains an image by fluorescence generated by emitting excitation light. In the fluorescence observation, it is possible to irradiate a body tissue with excitation light to observe the fluorescence from the body tissue (autofluorescence observation), or to locally inject a reagent such as indocyanine green (ICG) into a body tissue and irradiate the body tissue with excitation light corresponding to the fluorescence wavelength of the reagent to obtain a fluorescent image, for example. The light source device 5157 may be able to supply narrow band light and/or excitation light corresponding to such special light observation.

(Camera Head and CCU)

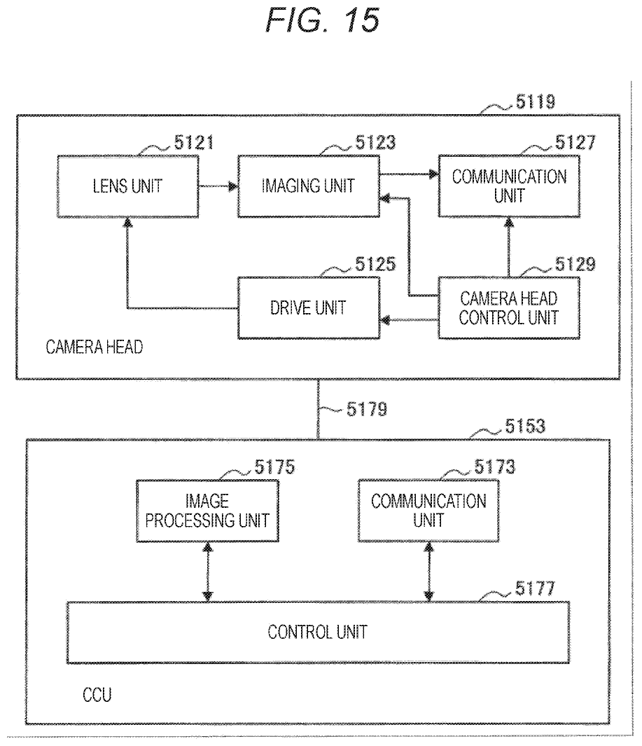

The functions of the camera head 5119 and the CCU 5153 of the endoscope 5115 will be described in more detail with reference to FIG. 15. FIG. 15 is a block diagram illustrating an example of a functional configuration of the camera head 5119 and the CCU 5153 illustrated in FIG. 14.

Referring to FIG. 15, the camera head 5119 includes, as its functions, a lens unit 5121, an imaging unit 5123, a drive unit 5125, a communication unit 5127, and a camera head control unit 5129. Furthermore, the CCU 5153 includes, as its functions, a communication unit 5173, an image processing unit 5175, and a control unit 5177. The camera head 5119 and the CCU 5153 are communicably connected to each other in both directions by a transmission cable 5179.

First, the functional configuration of the camera head 5119 will be described. The lens unit 5121 is an optical system provided at a connection portion with the lens barrel 5117. The observation light captured from the distal end of the lens barrel 5117 is guided to the camera head 5119 and is incident on the lens unit 5121. The lens unit 5121 includes a combination of a plurality of lenses including a zoom lens and a focus lens. Optical characteristics of the lens unit 5121 are adjusted so that the observation light is focused on the light-receiving surface of the imaging element of the imaging unit 5123. Furthermore, positions on the optical axis of the zoom lens and the focus lens are movable to adjust the magnification and focus of a captured image.

The imaging unit 5123 includes an imaging element, and is arranged at the subsequent stage of the lens unit 5121. The observation light passing through the lens unit 5121 is focused on the light-receiving surface of the imaging element, and an image signal corresponding to the observation image is generated by photoelectric conversion. The image signal generated by the imaging unit 5123 is provided to the communication unit 5127.

As the imaging element constituting the imaging unit 5123, for example, an element is used that is a complementary metal oxide semiconductor (CMOS) type image sensor, and is capable of color imaging having a Bayer array. Note that, as the imaging element, an element may be used compatible with imaging of the high-resolution image of greater than or equal to 4K, for example. The image of the surgical portion is obtained with high resolution, whereby the surgeon 5181 can grasp a state of the surgical portion in more detail, and can perform the surgery more smoothly.

Furthermore, the imaging element constituting the imaging unit 5123 includes a pair of imaging elements for acquiring image signals for the right-eye and left-eye to cope with 3D display. By performing the 3D display, the surgeon 5181 can grasp the depth of living tissue in a surgical portion more accurately. Note that, in a case where the imaging unit 5123 includes a multi-chip imaging element, a plurality of systems of the lens units 5121 is provided corresponding to respective imaging elements.

Furthermore, the imaging unit 5123 is not necessarily provided in the camera head 5119. For example, the imaging unit 5123 may be provided inside the lens barrel 5117 immediately after the objective lens.

The drive unit 5125 includes an actuator and moves the zoom lens and the focus lens of the lens unit 5121 by a predetermined distance along the optical axis by control of the camera head control unit 5129. As a result, the magnification and the focus of the captured image by the imaging unit 5123 can be appropriately adjusted.

The communication unit 5127 includes a communication device for transmitting/receiving various types of information to/from the CCU 5153. The communication unit 5127 transmits the image signal obtained from the imaging unit 5123 as RAW data to the CCU 5153 via the transmission cable 5179. At this time, to display the captured image of the surgical portion with low latency, the image signal is preferably transmitted by optical communication. This is because it is required that a video image of the surgical portion is displayed in real time as much as possible for safer and more reliable surgery since the surgeon 5181 performs the surgery while observing a state of the affected part with the captured image during the surgery. In a case where optical communication is performed, the communication unit 5127 is provided with a photoelectric conversion module that converts an electric signal into an optical signal. The image signal is converted into an optical signal by the photoelectric conversion module, and then transmitted to the CCU 5153 via the transmission cable 5179.

Furthermore, the communication unit 5127 receives the control signal for controlling the drive of the camera head 5119 from the CCU 5153. The control signal includes information regarding imaging conditions, for example, information that specifies the frame rate of the captured image, information that specifies the exposure value at the time of imaging, information that specifies the magnification and focus of the captured image, and/or other information. The communication unit 5127 provides the received control signal to the camera head control unit 5129. Note that, the control signal from the CCU 5153 may also be transmitted by optical communication. In this case, the communication unit 5127 is provided with a photoelectric conversion module that converts an optical signal into an electric signal, and the control signal is converted into an electric signal by the photoelectric conversion module and then provided to the camera head control unit 5129.

Note that, the above-described imaging conditions such as the frame rate, the exposure value, the magnification, and the focus are automatically set by the control unit 5177 of the CCU 5153 on the basis of the image signal acquired. That is, a so-called auto exposure (AE) function, auto-focus (AF) function, and auto white balance (AWB) function are installed in the endoscope 5115.

The camera head control unit 5129 controls the drive of the camera head 5119 on the basis of the control signal from the CCU 5153 received via the communication unit 5127. For example, the camera head control unit 5129 controls drive of the imaging element of the imaging unit 5123 on the basis of the information that specifies the frame rate of the captured image and/or the information that specifies the exposure at the time of imaging. Furthermore, for example, the camera head control unit 5129 appropriately moves the zoom lens and focus lens of the lens unit 5121 via the drive unit 5125 on the basis of the information that specifies the magnification and focus of the captured image. The camera head control unit 5129 may further have a function of storing information for identifying the lens barrel 5117 and the camera head 5119.

Note that, the camera head 5119 can be made to have resistance to autoclave sterilization by arranging the lens unit 5121, the imaging unit 5123, and the like in a sealed structure with high airtightness and waterproofness.

Next, the functional configuration of the CCU 5153 will be described. The communication unit 5173 includes a communication device for transmitting/receiving various types of information to/from the camera head 5119. The communication unit 5173 receives the image signal transmitted from the camera head 5119 via the transmission cable 5179. At this time, as described above, the image signal can be suitably transmitted by optical communication. In this case, to be adaptable to optical communication, the communication unit 5173 is provided with a photoelectric conversion module that converts an optical signal into an electric signal. The communication unit 5173 provides the image signal converted into the electric signal to the image processing unit 5175.

Furthermore, the communication unit 5173 transmits the control signal for controlling the drive of the camera head 5119 to the camera head 5119. The control signal may also be transmitted by optical communication.

The image processing unit 5175 performs various types of image processing on the image signal that is RAW data transmitted from the camera head 5119. Examples of the image processing includes various types of known signal processing, for example, development processing, image quality enhancement processing (such as band enhancement processing, super-resolution processing, noise reduction (NR) processing and/or camera shake correction processing), and/or enlargement processing (electronic zoom processing), and the like. Furthermore, the image processing unit 5175 performs detection processing on the image signal for performing AE, AF, and AWB.

The image processing unit 5175 includes a processor such as a CPU or GPU, and the image processing and detection processing described above can be performed by the processor operating in accordance with a predetermined program. Note that, in a case where the image processing unit 5175 includes a plurality of GPUs, the image processing unit 5175 appropriately divides information related to the image signal and performs the image processing in parallel by the plurality of GPUs.

The control unit 5177 performs various types of control regarding imaging of the surgical portion by the endoscope 5115 and display of the captured image. For example, the control unit 5177 generates the control signal for controlling the drive of the camera head 5119. At this time, in a case where the imaging condition is input by the user, the control unit 5177 generates the control signal on the basis of the input by the user. Alternatively, in a case where the AE function, the AF function, and the AWB function are installed in the endoscope 5115, the control unit 5177 generates the control signal by appropriately calculating the optimum exposure value, focal length, and white balance depending on a result of the detection processing by the image processing unit 5175.

Furthermore, the control unit 5177 causes the display device 5155 to display the image of the surgical portion on the basis of the image signal subjected to the image processing by the image processing unit 5175. At this time, the control unit 5177 recognizes various objects in the surgical portion image by using various image recognition technologies. For example, the control unit 5177 detects color, a shape of an edge, and the like of the object included in the surgical portion image, thereby being able to recognize the surgical tools such as the forceps, a specific body part, bleeding, mist at the time of using the energy treatment tool 5135, or the like. Furthermore, the image processing unit 5175 includes the configuration of the display processing unit 15 illustrated in FIG. 1, and performs peaking processing so that focus confirmation can be easily performed. The control unit 5177 sets the size and the position of the peaking area similarly to the control unit 20 of FIG. 1. When causing the display device 5155 to display the surgical portion image, the control unit 5177 causes the display device 5155 to superimpose and display various types of surgery assistance information on the surgical portion image by using the recognition result. The surgery assistance information is superimposed and displayed, and presented to the surgeon 5181, whereby the surgery can be performed more safely and reliably. Moreover, by presenting the surgical portion image on which the peaking display is performed, focusing on the surgical portion is facilitated. The surgical portion image on which the peaking display is performed may be presented on the display device 5155 referred to by the surgeon 5181, or instead of the display device 5155, may be displayed on a display device referred to by an assistant assisting the surgeon 5181, for example, the scopist who performs the focus adjustment operation on the camera head 5119 of the endoscope 5115. In this case, the peaking display does not hinder recognition of the surgical portion by the surgeon 5181. Furthermore, since the surgical portion image on which the peaking display is performed is presented on the display device referred to by the scopist, the scopist uses the peaking display to perform focus adjustment, so that the focused surgical portion image can be displayed on the display device 5155. Moreover, by setting the peaking area depending on the operation of the scopist, the surgeon 5181 can concentrate on the surgery.

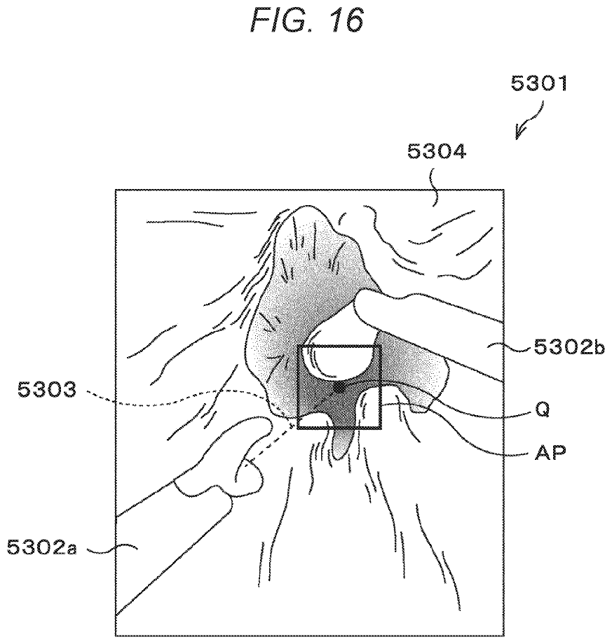

Furthermore, the setting of the peaking area may be automatically performed on the basis of the subject recognition result using the surgical portion image. For example, the peaking area is set by using the method disclosed in Japanese Patent Application Laid-Open No. 2015-228955. Specifically, a position in the three-dimensional space of the forceps in the surgical portion image is specified, and the intersection of an extension line extending the posture of the forceps and a surgical portion surface in the three-dimensional space is set as a target point. Moreover, an image area of a predetermined size based on the target point is set as a peaking area. FIG. 16 illustrates a peaking area setting example. In a surgical portion image 5301, two forceps 5302a and 5302b are imaged, and for example, the intersection point of an extension line 5303 extending the posture of the forceps 5302a and a surgical portion surface 5304 in the three-dimensional space is set as a target point Q. A rectangular area of a predetermined size centered on the target point Q is set as the peaking area AP.

If the peaking area is set in this way, even if the scopist does not set the peaking area depending on the progress of the surgery, the position of the peaking area can be automatically set at the optimum position depending on the progress of the surgery.

The transmission cable 5179 connecting the camera head 5119 and the CCU 5153 together is an electric signal cable adaptable to communication of electric signals, an optical fiber adaptable to optical communication, or a composite cable thereof.

Here, in the illustrated example, communication is performed by wire using the transmission cable 5179, but communication between the camera head 5119 and the CCU 5153 may be performed wirelessly. In a case where the communication between the two is performed wirelessly, it is not necessary to install the transmission cable 5179 in the operation room, so that a situation can be eliminated where the movement of the medical staff in the operation room is hindered by the transmission cable 5179.

In the above, the example has been described of the operation room system 5100 to which the technology according to the present disclosure can be applied. Note that, here, as an example, the case has been described where the medical system to which the operation room system 5100 is applied is the endoscopic surgical system 5113, but the configuration of the operation room system 5100 is not limited to such an example. For example, the operation room system 5100 may be applied to an inspection flexible endoscope system or a microscopic surgical system instead of the endoscopic surgical system 5113.

The series of processing steps described in the specification can be executed by hardware, software, or a combination of both. In a case where processing by software is executed, a program recording a processing sequence is installed in a memory in a computer incorporated in dedicated hardware and executed. Alternatively, the program can be installed and executed in a general-purpose computer capable of executing various types of processing.

For example, the program can be recorded in advance in Read Only Memory (ROM), a Solid State Drive (SSD) or a hard disk as a recording medium. Alternatively, the program can be temporarily or permanently stored (recorded) in a removable recording medium such as a flexible disk, compact disc read only memory (CD-ROM), magneto optical (MO) disk, digital versatile disc (DVD), Blu-ray Disc (registered trademark) (BD), magnetic disk, or semiconductor memory card. Such a removable recording medium can be provided as so-called packaged software.

Furthermore, the program may be transferred wirelessly or by wire to the computer from a download site through the network such as a local area network (LAN) or the Internet, besides being installed from the removable recording medium to the computer. The computer can receive the program transmitted in that way, and install the program in the recording medium such as a built-in hard disk.

Note that, the advantageous effects described in this specification are merely examples, and the advantageous effects of the present technology are not limited to them and may include additional effects that are not described herein. Furthermore, the present technology should not be interpreted to be limited to the embodiments of the technology described above. The embodiments of the present technology disclose the present technology through examples, and it should be obvious that those skilled in the art can modify or replace those embodiments with other embodiments without departing from the scope of the technology. In other words, the claims should be taken into account in understanding the subject matter of the present technology.

Furthermore, the image processing device of the present technology can have the following configuration.

(1) An image processing device including:
a display processing unit that performs highlighting processing on the basis of an edge component detected by using an image signal; and
a processing area setting unit that sets at least a size or a position of a processing target area on which the highlighting processing is performed.

(2) The image processing device according to (1), further including
an operation unit that generates an operation signal depending on a user operation, in which
the processing area setting unit sets the processing target area depending on the user operation.

(3) The image processing device according to (2), in which
the operation unit includes an operation key, and
the processing area setting unit sets the size of the processing target area depending on a size setting operation using the operation key, and sets the position of the processing target area depending on a position setting operation using the operation key.

(4) The image processing device according to (2), in which
the operation unit includes a touch panel provided on a display surface of a display unit that displays an image based on the image signal, and
the processing area setting unit sets the size and the position of the processing target area on the basis of a start position and an end position of the user operation on the touch panel.

(5) The image processing device according to (4), in which the processing area setting unit sets the size of the processing target area on the basis of the end position with the start position as a reference of the position of the processing target area.

(6) The image processing device according to any of (1) to (5), in which the processing area setting unit performs setting in the processing target area on the basis of a predetermined subject detected by subject recognition using the image signal.

(7) The image processing device according to (6), in which the processing area setting unit sets, as the processing target area, an area including the whole of the predetermined subject detected.

(8) The image processing device according to (6), in which the processing area setting unit sets the position of the processing target area on the basis of a posture of the predetermined subject detected.

(9) The image processing device according to any of (6) to (8), in which the processing area setting unit sets the predetermined subject depending on an imaging scene mode when the image signal is generated.

(10) The image processing device according to any of (1) to (9), in which the display processing unit performs the highlighting processing in a case where a value based on the edge component is greater than or equal to a predetermined value, and
the predetermined value is set on the basis of the processing target area.

(11) The image processing device according to any of (1) to (9), in which the display processing unit detects the edge component by filter processing, and
a filter used for the filter processing is set on the basis of the processing target area.

(12) The image processing device according to any of (1) to (11), in which the display processing unit changes a signal level inside or outside the processing target area.

(13) The image processing device according to any of (1) to (12), in which the display processing unit changes a signal used for replacement in the highlighting processing depending on a current focal position with respect to a focus position.

(14) The image processing device according to any of (1) to (12), in which the display processing unit changes a signal used for replacement in the highlighting processing depending on the edge component.

(15) The image processing device according to any of (1) to (12), in which the display processing unit changes a color of a signal used for replacement in the highlighting processing depending on a color inside the processing target area.

(16) The image processing device according to any of (1) to (15), in which the display processing unit performs the highlighting processing at a predetermined cycle.

(17) The image processing device according to any of (1) to (16), further including an output unit that outputs, to an external device, the image signal subjected to the highlighting processing in the display processing unit.

### INDUSTRIAL APPLICABILITY

According to the image processing device, the image processing method, and the program of the present technology, the highlighting processing is performed on the basis of the edge component detected by using the image signal. Furthermore, setting is performed of at least the size or the position of the processing target area on which the highlighting processing is performed. Thus, the peaking processing can be performed in a desired image area, and focus confirmation can be easily performed. Thus, the present technology is suitable for a system in which a captured image with high focus accuracy is required, for example, a surgical system or the like.

### REFERENCE SIGNS LIST

10 Imaging device
11 Imaging optical system block
12 Imaging unit
13 Camera processing unit
14 Recording/reproducing unit
15 Display processing unit
16 Display unit
17 Output unit
18 Operation unit
20 Control unit
151 Edge detection unit
152 Highlighting processing unit

### 181 Selection key

### 182 Enter key

1. An image processing device comprising:
a display processing unit that performs highlighting processing on a basis of an edge component detected by using an image signal; and a processing area setting unit that sets at least a size or a position of a processing target area on which the highlighting processing is performed.

2. The image processing device according to claim 1, further comprising
an operation unit that generates an operation signal depending on a user operation, wherein
the processing area setting unit sets the processing target area depending on the user operation.

3. The image processing device according to claim 2, wherein
the operation unit includes an operation key, and
the processing area setting unit sets the size of the processing target area depending on a size setting operation using the operation key, and sets the position of the processing target area depending on a position setting operation using the operation key.

4. The image processing device according to claim 2, wherein
the operation unit includes a touch panel provided on a display surface of a display unit that displays an image based on the image signal, and
the processing area setting unit sets the size and the position of the processing target area on a basis of a start position and an end position of the user operation on the touch panel.

5. The image processing device according to claim 4, wherein
the processing area setting unit sets the size of the processing target area on a basis of the end position with the start position as a reference of the position of the processing target area.

6. The image processing device according to claim 1, wherein
the processing area setting unit performs setting in the processing target area on a basis of a predetermined subject detected by subject recognition using the image signal.

7. The image processing device according to claim 6, wherein
the processing area setting unit sets, as the processing target area, an area including a whole of the predetermined subject detected.

8. The image processing device according to claim 6, wherein
the processing area setting unit sets the position of the processing target area on a basis of a posture of the predetermined subject detected.

9. The image processing device according to claim 6, wherein
the processing area setting unit sets the predetermined subject depending on an imaging scene mode when the image signal is generated.

10. The image processing device according to claim 1, wherein
the display processing unit performs the highlighting processing in a case where a value based on the edge component is greater than or equal to a predetermined value, and
the predetermined value is set on a basis of the processing target area.

11. The image processing device according to claim 1, wherein
the display processing unit detects the edge component by filter processing, and
a filter used for the filter processing is set on a basis of the processing target area.

12. The image processing device according to claim 1, wherein
the display processing unit changes a signal level inside or outside the processing target area.

13. The image processing device according to claim 1, wherein
the display processing unit changes a signal used for replacement in the highlighting processing depending on a current focal position with respect to a focus position.

14. The image processing device according to claim 1, wherein
the display processing unit changes a signal used for replacement in the highlighting processing depending on the edge component.

15. The image processing device according to claim 1, wherein
the display processing unit changes a color of a signal used for replacement in the highlighting processing depending on a color inside the processing target area.

16. The image processing device according to claim 1, wherein
the display processing unit performs the highlighting processing at a predetermined cycle.

17. The image processing device according to claim 1, further comprising
an output unit that outputs, to an external device, the image signal subjected to the highlighting processing in the display processing unit.

18. An image processing method comprising:
performing highlighting processing, by a display processing unit, on a basis of an edge component detected by using an image signal, and
performing setting, by a processing area setting unit, of at least a size or a position of a processing target area on which the highlighting processing is performed.

19. A program that causes a computer to execute processing using an image signal,
the program causing the computer to execute:
a procedure of performing highlighting processing on a basis of an edge component detected by using an image signal; and
a procedure of setting at least a size or a position of a processing target area on which the highlighting processing is performed.
